(19) United States
(12) Patent Application Publication
(10) Pub. No.: US 2007/0191691 A1
(43) Pub. Date: Aug. 16, 2007

(54) Identification of guilty knowledge and malicious intent

(76) Inventor: Martin Polanco, San Diego, CA (US)

Correspondence Address: FISH & RICHARDSON, PC, P.O. BOX 1022, MINNEAPOLIS, MN, 55440-1022, US

(21) Appl. No.: 11/436,289

(22) Filed: May 17, 2006

Related U.S. Application Data
(60) Provisional application No. 60/683,029, filed on May 19, 2005.

(51) Int. Cl. A61B 5/00; A61B 6/00; A61B 5/04; A61B 13/00

(52) U.S. Cl. 600/301; 600/544; 600/558; 600/559; 600/473

(57) ABSTRACT

Guilty knowledge or malicious intent can be identified in a subject by presenting a stimulus to the subject, detecting an electrical potential at a location on the skin of the subject, detecting infrared light reflected from the brain of the subject, and analyzing the detected electrical potential and the detected infrared light to identify an indication of guilty knowledge or malicious intent. Also, an identified indication of guilty knowledge or malicious intent can be associated with the presented stimulus. Guilty knowledge or malicious intent further can be identified by detecting one or more muscle movements comprising a facial expression and analyzing the one or more detected muscle movements to identify a visible indication of guilty knowledge or malicious intent, which also can be associated with the presented stimulus. Additionally, the stimulus can comprise one or more of a question, an image, a video clip, a sound, and an audio clip.

### CROSS-REFERENCE TO RELATED APPLICATION

This application claims priority to and the benefit of U.S. Provisional Patent Application Ser. No. 60/683,029, filed May 19, 2005, entitled IDENTIFICATION OF GUILTY KNOWLEDGE AND MALICIOUS INTENT, the entire disclosure of which is hereby incorporated by reference.

### BACKGROUND

The present disclosure relates to devices and systems for detecting guilty knowledge and malicious intent, and to stimuli employed in conjunction with such devices.

Society has long sought to automate the detection of guilty knowledge and malicious intent. In the mid 19th century, Cesare Lombroso mechanized a method for recording the blood-pressure and pulse of a human subject. The method was employed as a means of assessing the honesty of criminals. In 1921, John Larson modified the method to account for respiration rate. Leonard Keeler further expanded the method in 1939 through the addition of skin resistance measurements and a direct-coupled solid state amplifier. Together, these inputs form the basis of the polygraph.

Under stressful conditions or when a person is trying to remove themselves from a potentially harmful situation, subconscious psychological events typically arise. These psychological events are further accompanied by physical manifestations that are largely involuntary. Psychophysiology is an area of psychology that utilizes subtle changes in physiological functions, such as skin resistance, heart rate, and blood pressure, which are controlled by the autonomic nervous system, to differentiate between a variety of psychological states. For example, sweat glands can release a burst of liquid under certain conditions, which results in an increase in the skin's galvanic response. The change in skin conductance is measurable and can indicate the mental state of the subject. Studies of the polygraph indicate that the skin conductance response is the most important signal for detecting deception because it provides a reliable indication of anxiety and it is involuntary. Nonetheless, this measure can only directly detect increased anxiety and not deceit. Additional measurable physiological signals can provide further insight into a subject's mental state, including respiration volume, pulse rate, and blood flow. These functions are used because they are not under precise voluntary control and are not normally detectable by the person in whom they occur.

Multi-channel physiological recording, or polygraph, is currently the most widely used method for the detection of deception. But, the effectiveness of the polygraph in the detection of deception is limited by its reliance on the physical, and thus peripheral, manifestations of anxiety. Because conventional polygraphy relies on psycho-physiological measures of autonomic nervous system response in order to detect the anxiety associated with guilt or lying, it is difficult to differentiate guilt from anxiety. As such, the test produces an unacceptably high level of false positive results.

In a conventional polygraph test, emotion-driven physiological responses to questions that are relevant to the circumstances under investigation are compared with responses to control questions. The control questions are invasive, personal questions that are not relevant to the issue at hand and are designed to be emotionally and physiologically disturbing to the subject. A greater response to the relevant questions leads to a determination that the subject is trying to deceive the examiner and is therefore guilty. Conversely, a greater response to the control questions leads to a determination that the subject is not attempting to deceive the examiner and is therefore innocent. In an attempt to avoid a false positive result, the examiner must ask penetrating questions during the pre-test interview to identify personal material that is sufficiently disturbing and stress-producing to produce effective control questions.

To elicit a stress response to the control questions during the test, the examiner typically deceives the subject, leading him to believe that a large response to the control questions will make him appear guilty or deceptive, rather than innocent or non-deceptive. The examiner's strategy is instrumental in producing the desired response. Thus, in conventional polygraphy, innocent subjects are deceived and subjected to a highly invasive and stressful situation both during the pre-test interview and during the test.

### SUMMARY

The present inventor recognized the need to implement devices and systems for the detection of guilty knowledge and malicious intent that are accurate and reduce the number of false positives. The present inventor also recognized the need to implement devices and systems for the detection of guilty knowledge and malicious intent that can incorporate multiple test methodologies. Accordingly, the techniques and apparatus described here implement tests for directly identifying the presence of guilty knowledge and malicious intent in a human subject.

In general, in one aspect, the techniques can be implemented to include presenting a stimulus to a subject; detecting an electrical potential at a location on the skin of the subject; detecting infrared light reflected from the brain of the subject; and analyzing the detected electrical potential and the detected infrared light to identify an indication of guilty knowledge or malicious intent.

The techniques also can be implemented to include associating the identified indication of guilty knowledge or malicious intent with the presented stimulus. The techniques further can be implemented such that the presented stimulus comprises a control question. Additionally, the techniques can be implemented to include detecting one or more muscle movements comprising a facial expression and analyzing the one or more detected muscle movements to identify a visible indication of guilty knowledge or malicious intent. Further, the techniques can be implemented to include associating the identified visible indication of guilty knowledge or malicious intent with the presented stimulus.

The techniques also can be implemented such that the stimulus comprises one or more of a question, an image, a video clip, a sound, and an audio clip. Additionally, the techniques can be implemented such that detecting an electrical potential further comprises detecting a sensed electroencephalograph potential corresponding to a predetermined frequency band. Further, the techniques can be implemented to include analyzing an amplitude associated with the sensed electroencephalograph potential and identifying a P-300 wave form.

The techniques also can be implemented such that analyzing reflected infrared light further comprises determining an amount of reflected infrared light corresponding to one or more wavelengths and identifying a change in blood oxygenation based on the determined amount. Additionally, the techniques can be implemented to include identifying a change in total blood volume based on the determined amount. Further, the techniques can be implemented to include presenting an additional stimulus to the subject based on the identified indication of guilty knowledge or malicious intent; detecting an electrical potential associated with the additional stimulus; detecting reflected infrared light associated with the additional stimulus; and analyzing the detected electrical potential and the detected infrared light to identify an indication of guilty knowledge or malicious intent associated with the additional stimulus.

In general, in another aspect, the techniques can be implemented to include a stimulus presentation device; a human wearable device including an EEG sensor and a light detector, wherein the human wearable device is configured to detect an electrical potential at a location on the skin of a subject and to detect infrared light reflected from the brain of the subject; and a data processing device, wherein the data processing device is configured to analyze the detected electrical potential and the detected infrared light to identify an indication of guilty knowledge or malicious intent.

The techniques also can be implemented such that the stimulus presentation device comprises one or more of a speaker and a visible display. The techniques further can be implemented such that the presentation device is physically coupled to the human wearable device. Additionally, the techniques can be implemented such that the human wearable device comprises a helmet.

The techniques also can be implemented such that the human wearable device further comprises a light source comprised of a plurality of light emitting diodes, wherein two or more of the light emitting diodes produce light of different wavelengths; and a modulator configured to modulate the wavelengths of light produced by the plurality of light emitting diodes. Further, the techniques can be implemented to include a camera configured to capture a facial movement of the subject. Additionally, the techniques can be implemented such that the data processing device is further configured to analyze the captured facial movement to identify a visible indication of guilty knowledge or malicious intent.

The techniques also can be implemented to include a data entry device configured to receive an input from the subject. Further, the techniques can be implemented such that the data entry device comprises one or more of a keyboard, a mouse, a touch pad, a touch screen, a key pad, a microphone, a joystick, a button, a switch, a card reader, a scanner, and a biometric identification device.

The techniques described in this specification can be implemented to realize one or more of the following advantages. For example, the techniques can be implemented to accurately identify the existence of guilty knowledge or malicious intent in a wide-variety of human subjects. The techniques also can be implemented using a portable device to permit testing in public locations. Additionally, the techniques can be implemented to permit the use of automated stimuli presentation. Further, the techniques can be implemented to permit the detection of deceptive responses without relying upon invasive personal questions. The techniques also can be implemented to permit the placement of terminals in a wide-variety of locations, comprising either self-service or attendant monitored systems. Further, the techniques can be implemented to use more direct measures of the neural activity associated with deception and lying, thus permitting more accurate detection of guilty knowledge and malicious intent. Additionally, the techniques can be implemented to provide increased privacy for travelers.

These general and specific techniques can be implemented using an apparatus, a method, a system, or any combination of an apparatus, methods, and systems. The details of one or more implementations are set forth in the accompanying drawings and the description below. Further features and advantages will become apparent from the description, the drawings, and the claims.

### BRIEF DESCRIPTION OF THE DRAWINGS

FIG. 1 presents a portable system for the detection of guilty knowledge.

FIG. 2 depicts sensor placement corresponding to a human skull.

FIG. 3 depicts a headband including functional Near Infrared (fNIR) sensors.

FIG. 4 presents a kiosk system for the detection of guilty knowledge.

FIG. 5 presents a flowchart for conducting a Guilty Knowledge Test.

FIG. 6 depicts a P-300 brain wave.

FIG. 7 depicts fNIR sensor results.

FIG. 8 is a flowchart describing a process for identifying guilty knowledge or malicious intent in a subject.

Like reference symbols indicate like elements throughout the specification and drawings.

### DETAILED DESCRIPTION

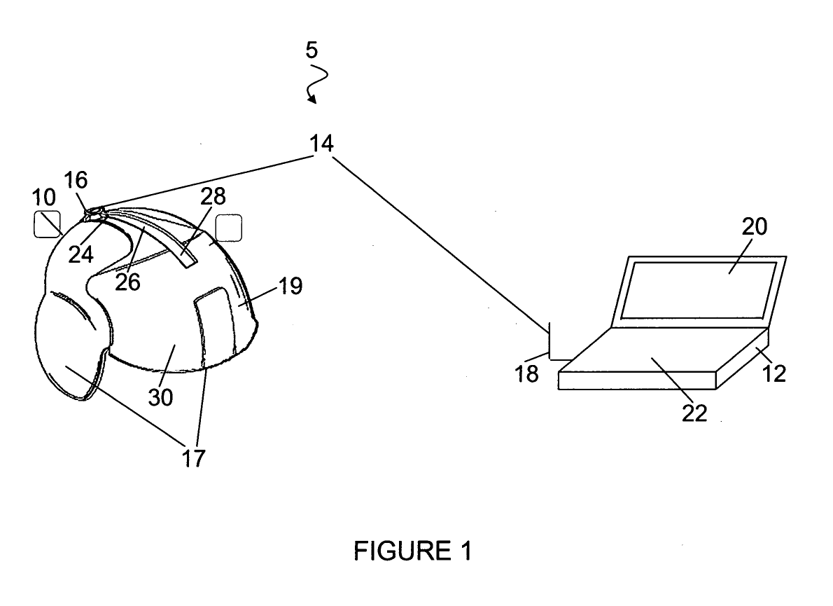

FIG. 1 presents a portable system 5 for the detection of guilty knowledge and malicious intent. In an implementation, the system 5 includes a human wearable data collection device, such as a helmet 10, and a data processing device 12, such as a notebook computer, a hand-held computer, or other general or special purpose computing device. The helmet 10 and the data processing device 12 can be configured to communicate bi-directionally through a wireless interface 14. The wireless interface 14 can be any interface known in the art, including radio frequency and infrared. Additionally, the communications over the wireless interface can be encrypted for data security. The helmet 10 can transmit and receive data over the wireless interface 14 through a wireless adapter 16. Similarly, the data processing device 12 can transmit and receive data over the wireless interface 14 through a wireless adapter 18.

The helmet 10 can receive stimulus and calibration information from the data processing device 12 through the wireless interface 14. Similarly, the data processing device 12 can receive information, such as sensor data collected from one or more sensors included in the helmet 10, which also can be transmitted over the wireless interface 14. Because it can be advantageous for the data processing device 12 to transmit data to the helmet 10 while the helmet 10 is transmitting data to the data processing device 12, the helmet 10 and the data processing device 12 can be configured to communicate in full duplex (simultaneously). In another implementation, the helmet 10 and the data processing device 12 can be configured to communicate bi-directionally using a wired-interface, such as one or more cables.

In addition to providing information to the helmet 10 and receiving information from the helmet 10, the data processing device 12 also can be configured to display information on one or more display devices 20, such as a display screen, monitor, or printer. For example, in an implementation, the data processing device 12 can provide an indication to a user, such as an examiner, that corresponds to one or more events detected by a sensor included in the helmet 10. Further, the data processing device 12 also can be configured to present stimulus information, such as one or more questions, commands, visual images, sounds, or audio clips, to a subject. Additionally, the data processing device 12 can include one or more input devices 22 to receive input from a user, such as commands and responses. The one or more input devices 22 can include any input device known in the art, such as a keyboard, a mouse, a touch pad, a keypad, a microphone and speech recognition processor, a touch screen, and a joystick. In an implementation, the data processing device 12, or a portion thereof, can be included in the helmet 10.

The helmet 10 also can include one or more sensors for detecting the psychophysical responses of a subject to presented stimuli. An electroencephalograph (EEG) measures and records brainwave activity by sensing spontaneous electrical potential of a person's scalp, cortex, or cerebrum at one or more sites. Each EEG channel corresponds to a particular electrode combination that is in contact with the subject. The sensed EEG potential at each channel is amplified by a differential amplifier, and the amplifier output signal can be converted into a readable output.

EEG signals exhibit different frequencies depending upon varying activity. The EEG signal frequencies are classified into four basic frequency bands, which are generally referred to as “Delta” (0-3.5 Hertz); “Theta” (4-8 Hertz); “Alpha” (8-13 Hertz); and “Beta” (13+Hertz). A neurologist can determine the predominant frequency of a particular channel during a particular time window by measuring the period the EEG signal wave form shown on the EEG record. This requires considerable training and is highly dependent upon the skill of the neurologist, since the EEG signal wave form typically includes multiple frequency components. EEG also can be driven by specific extrinsic or endogenous events. For example, a regularly occurring stimulus will elicit a series of waves each time it is presented. The entire series of waves is referred to as an event-related potential (ERP).

Besides the frequency of the EEG or ERP wave forms, the amplitude is often analyzed. Significance has been established when large amplitude brain waves occur at about 300 ms or more after the eliciting event. There is evidence to suggest that this P-300 (or P-3) wave process is invoked during the updating, or “refreshing”, of representations in working memory. Large P-300's may be elicited by rare or unexpected events, and/or when they are relevant to a task the subject is performing as brain activity is being recorded. Such events may lead to restructuring or updating of working memory, and this activity is part of the ongoing process of maintaining accurate schema of the environment. Because P-300 amplitude can indicate the degree of restructuring in working memory, P-300 amplitude also can be associated with a subsequent recall of information.

In view of the current knowledge of the frequency and amplitude of brain wave forms and with the advent of widespread use of the computer in behavioral neuroscience, the analysis of data has become easier. Often, it is desirable to have an objective method of determining whether a person has seen, experienced, or otherwise has knowledge of a particular item, such as a weapon, a crime scene, the configuration of a location or site, a document, an object, or a person's face. Such knowledge is what is taught by prior art procedures and devices used in “guilty knowledge” tests, a sub-category of procedures used in physiological detection of deception (“lie detection”).

If a discrete stimulus, such as a sound, a light flash, or a tap, for example, is presented to a subject, a corresponding electroencephalogram can show a series of time-locked responses or ERPs. For example, if a subject is presented with a series of stimuli of two types, e.g., a high tone and a low tone, and if either of those tones is presented in 20 of 100 trials (with the remaining 80 trials containing the other tone), the rare stimulus will evoke a large ERP referred to as a P-300 brain wave. The amplitude of the P-300-wave form rarely fluctuates.

U.S. Pat. No. 4,932,416, incorporated herein by reference, describes a method of evoking a P-300 wave form and utilizes this method in knowledge detection procedures including guilty knowledge detection, control question testing, and other “lie detection” and related procedures. Letters are used to describe the detection location, including the Frontal (F), Temporal (T), Central (C), Parietal (P) and Occipital (O) lobes. Additionally, numbers indicate the side of the hemisphere on which the electrode is placed. Even numbers indicate the right side of the brain and odd numbers indicate the left side of the brain. The letter z indicates an electrode placed on the mid-line of the brain.

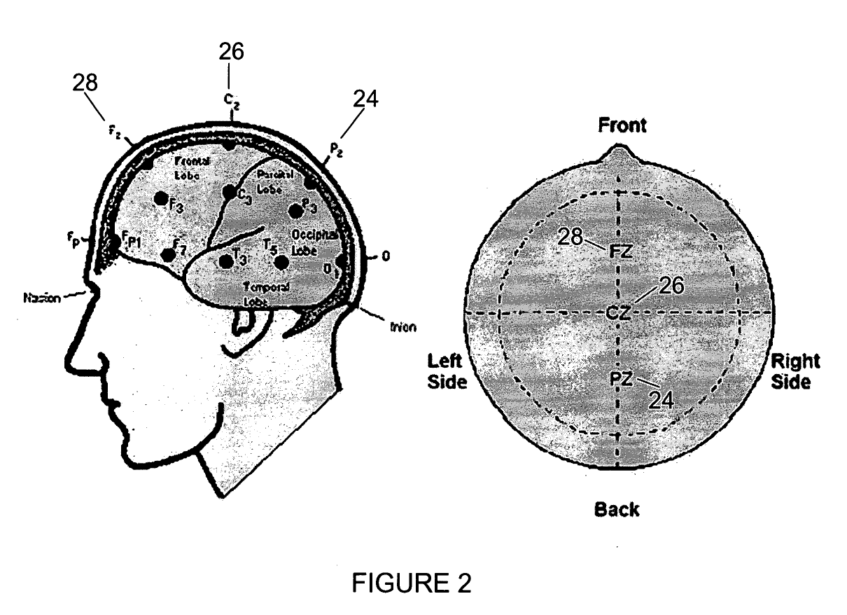

In an implementation, a plurality of EEG sensors can be used to record measurements in various locations on the scalp of a subject, such as Fz, Cz, and Pz. For example, an EEG sensor 24 corresponding to Pz, an EEG sensor 26 corresponding to Cz, and an EEG sensor corresponding to Fz 28 can be coupled to the interior of the helmet 10 along the central axis. The positions of the EEG sensors 24, 26, and 28, as related to the scalp of the subject, are further illustrated in FIG. 2. In another implementation, other EEG sensors can be used in conjunction with or in place of one or more of the EEG sensors 24, 26, and 28.

The plurality of EEG sensors can be used to develop a frequency-based map corresponding to the brain of a subject, which then can be used to assess the level of stress the subject is experiencing. As such, the data detected by one or more EEG sensors, such as the EEG sensors 24, 26, and 28 included in the helmet 10, can be used to identify one or more stress conditions that are likely to accompany and follow deception. In an implementation, the helmet 10 also can be shielded against external influences, such as electromagnetic interference.

Cognitive differences between lying and telling the truth also can be reflected in other correlators of brain activity, such as regional cerebral flow (rCBF). Unlike the ERP, the spatial resolution of blood oxygenation level dependent (BOLD) flow magnetic resonance imaging (fMRI) is sufficient to localize changes in rCBF that are related to regional neuronal activity during cognition. When a subject makes the decision to lie, and even before the lie is expressed, there is a burst of blood flow over a period of several milliseconds, which can be read by one or more sensors coupled to the subject. Optically tracking both the flow of blood or blood volume changes (BVCs), and variations in the oxygenation levels of the right prefrontal cortex of the brain, make this system much more accurate than traditional lie detector tests.

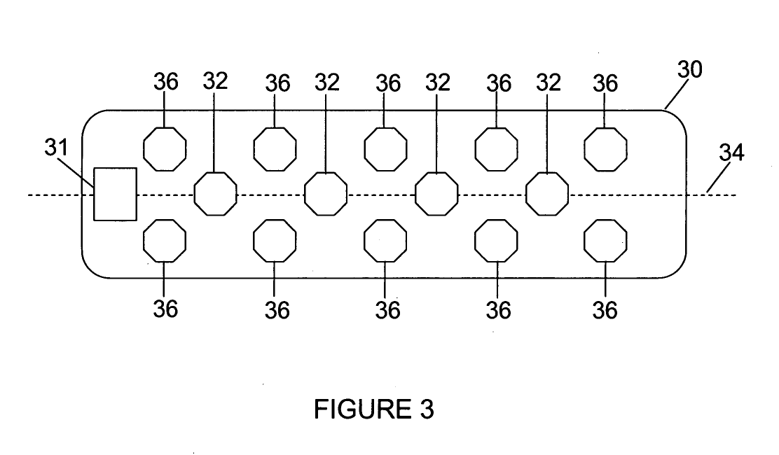

As such, the helmet 10 also can include one or more functional Near Infrared (fNIR) sensors, which can be included in a headband 30 or pad coupled to the interior of the helmet 10 and positioned relative to the subject's forehead. FIG. 3 presents an implementation of the headband 30, in which a plurality of light sources 32, such as gold-colored light sources, are attached along the central axis 34 of the headband 30. Each of the light sources 32 includes a plurality of infrared light-emitting diodes (LEDs) that produce light of differing wavelengths. One or more wavelengths can be selected such that they are absorbed more by oxygenated blood. Similarly, one or more other wavelengths can be selected such that they are absorbed more by deoxygenated blood. The headband 30 also can include a modulator 31 for modulating the LEDs included in the light sources 32. In an implementation, the modulator 31 can operate at one or more frequencies in order to modulate the wavelengths of light produced by the LEDs. For example, the modulator 31 can operate using a 3-kHz carrier and can thus modulate the LEDs between a wavelength of 730 nanometers and 850 nanometers.

The LEDs can be positioned in a specific alignment, such as an array, with a plurality of silicon light detectors 36 that are configured to detect infrared light reflected back from the brain. As light leakage from the light sources 32 to the silicon light detectors 36 may result in an incorrect reading or false positive, the light sources 32 and the silicon light detectors 36 should be situated such that they are in close contact with the skin of the subject. The plurality of silicon light detectors 36 can detect the presence of prefrontal brain activation through the detection of infrared light reflected from the brain and produce one or more signals indicating the amount of reflected infrared light that is detected. The one or more signals can be transmitted to the data processing device 12 over the wireless interface 14.

The data processing device 12 can be configured to analyze the reflected light signals detected by the plurality of silicon light detectors 36 to identify changes in total blood volume and in the level of blood oxygenation. The modulator 31 and the light sources 32 included in the headband 30 also can be controlled by the data processing device 12. The data processing device 12 can control the duration of the light bursts from the plurality of LEDs included in the light sources 32. For example, the LEDs can be used to produce 10-millisecond light bursts. Further, the data processing device 12 can process the signals produced by the silicon light detectors 36 in response to the detection of reflected infrared light. Additionally, the data processing device 12 can be configured to process the signals produced by the silicon light detectors 36 to eliminate any noise and then amplify and integrate the signals.

With reference to FIG. 1, the helmet 10 further can include one or more stimulus delivery mechanisms, such as speakers and image displays. For example, the helmet 10 can include one or more speakers 17 located proximally to one or both ears in order to deliver audio signals, such as questions and audio clips. Further, the helmet 10 can include a visual display 19 for the presentation of questions and visual stimulus, including images and video clips. The visual display 19 can be attached to the helmet, such as an integrated liquid crystal display. In such a configuration, the visual display 19 can be attached in a manner that permits it to be retracted when visual stimulus is not being provided. For example, the visual display 19 can be integrated into a visor or affixed to an arm, or similar pivoting mechanism, that can be rotated into a viewable position when visual stimulus is to be presented to a subject and can be rotated into a stored position (shown) when visual stimulus is not being presented.

In another implementation, the visual display can be separate from the helmet 10. For example, the visual display can comprise goggles or glasses that can be placed over the subject's eyes when a questions or visual stimulus is to be presented. Alternately, the visual display can comprise a television monitor, a computer screen, a projector, or the display device 20 associated with the data processing device 12.

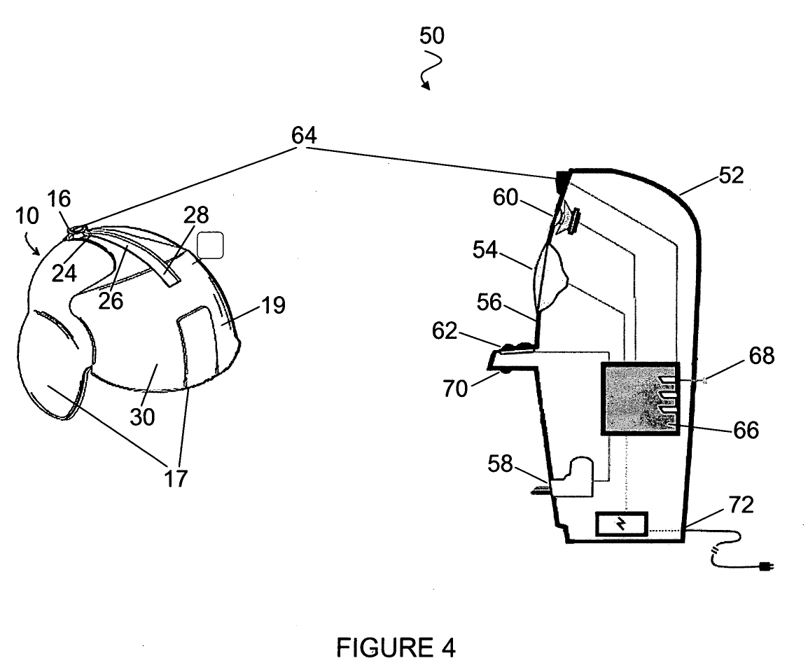

FIG. 4 presents an implementation of a kiosk system 50 for detecting guilty knowledge and malicious intent. A kiosk 52 can include a display 54 that is viewable through the kiosk enclosure 56. The kiosk 52 also can include a printer 58 and one or more speakers 60 for providing output, such as to a user. Further, the kiosk 52 can include one or more data entry devices 62 to permit a user to enter information, such as responses and commands. The one or more data entry devices 62 can include a keyboard, a mouse, a touch pad, a keypad, a microphone and speech recognition application, a touch screen, a joystick, and one or more buttons or switches. Additionally, the kiosk 52 can be configured to include one or more data interfaces 70, such as a universal serial bus (USB) connector, a parallel port, a serial port, and an IEEE 1394 “FireWire” port. An external data device, such as a computer or storage device, can be connected to the kiosk 52 through a data interface 70 to permit data to be uploaded to or downloaded from the kiosk 52.

Communications between the kiosk 52 and the helmet 10 can be carried out through a wireless interface 64, such as a bi-directional, full-duplex communication link. The wireless interface 64 is similar to the wireless interface 14 discussed above with reference to FIG. 1. In another implementation, communications between the kiosk 52 and the helmet 10 can be carried out through a wired interface, such as one or more cables. The kiosk 52 also can include a processor 66 that controls the operation of the kiosk 52. The processor 66 can be interconnected with one or more storage devices (not shown), such as random access memory, read-only memory, and a hard disk. The one or more storage devices can be configured to store instructions, such as application programs, that can be executed by the processor 66 and information that is received by the processor 66. For example, the processor 66 can be configured to execute properly entered commands and to coordinate the delivery of messages and stimulus to a user. The processor 66 further can be configured to receive and processor sensor information detected by the helmet 10. In the kiosk system 50, the helmet 10 can be configured as described with respect to the portable system 5.

The kiosk 52 also can include a network communications interface 68 to permit communications with one or more remote computing devices over a public network, such as the Internet, or a private network. The network communications interface 68 can be any network interface known in the art, such as a dial-up modem, a cable modem, a satellite modem, and a network interface card. Further, the kiosk 52 can be configured to derive power from a power adapter 72, such as an alternating current adapter. In an implementation, the one or more data entry devices 62 further can include a card reader, such as a magnetic-stripe card reader or a smart-card reader, and a scanner, such as a barcode scanner and a passport scanner. Further, the data entry devices 62 also can include a biometric identification device, such as a fingerprint scanner, a retinal scanner, and a facial scanner.

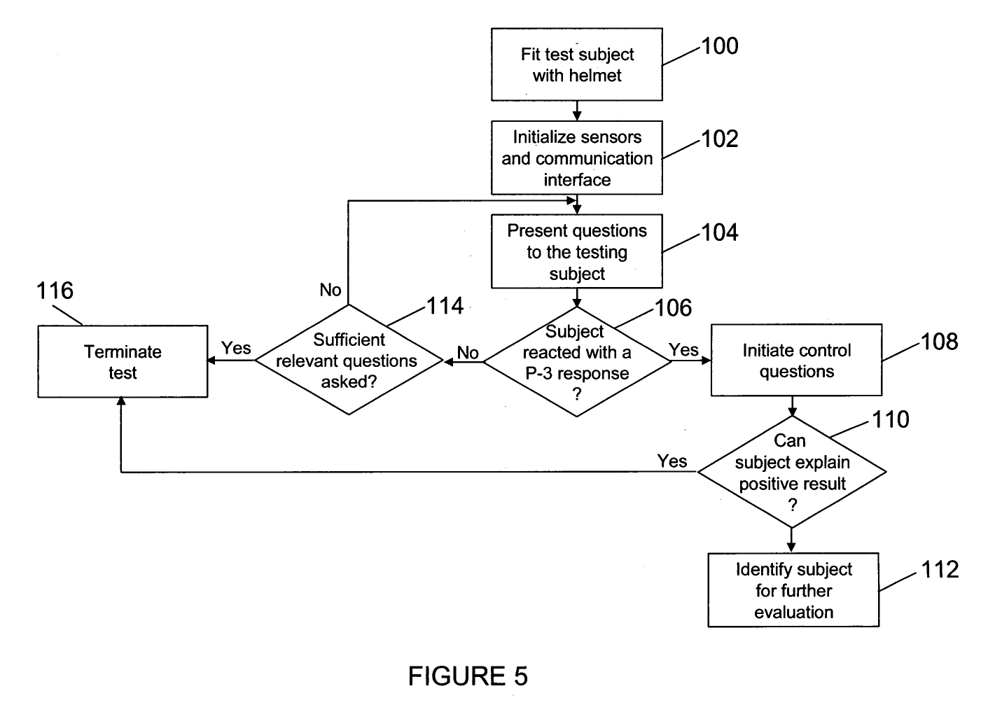

The helmet 10, in conjunction with either the data processing device 12 or the kiosk 52, can be used to screen subjects for the presence of guilty knowledge and malicious intent. FIG. 5 presents a flowchart describing a technique for conducting a test to detect the presence of guilty knowledge or malicious intent in a subject. The subject to be tested is fitted with the helmet 10 (100). After the subject is fitted with the helmet 10, the sensors and communication interface are initialized to ensure that they are functioning properly (102). A question presentation and timing application initiates an Event Related Potential Guilty Knowledge Test (ERP GKT) and presents one or more questions to the subject (104). The questions can be conventional questions designed to elicit a response in the form of information. For example, a conventional question may ask the subject to identify whether he has ever been convicted of a felony. The questions also can be unconventional questions, such as an image or audio segment that seeks to identify recognition in the subject, which are designed to evoke a psycho-physiological response.

In an implementation, one or more images, video segments, and audio segments are presented to the user before, after, or in conjunction with a plurality of multiple choice questions, or in place of the plurality of multiple choice questions. The one or more images, video segments, and audio segments include a mixture of relevant and irrelevant stimuli. For example, during an ERP GKT, many superficially similar stimuli are presented in sequence, with only a minority representing stimuli that the subject is believed to recognize. The data processing device 12 can be configured to detect electrical brain responses associated with each of the stimuli presented to a subject and analyze the electrical brain responses to identify one or more event related brain potentials.

As described above, the EEG sensors 24, 26, and 28 included in the helmet 10 can detect ERPs that occur during a test. The EEG sensors 24, 26, and 28 are monitored to determined whether the test subject reacts to any of the probes with a P-300 response (106). Thus, the results of the ERP GKT can be determined through mathematical analysis by a computer and do not rely upon subjective analysis. If the test subject reacts with a P-300 response, the control questions are initiated (108). The question presentation and timing application selects the question sequence to be applied depending on which of the EEG sensors 24, 26, and 28 detected the P-300 response.

As described above, one or more control questions can be presented to the subject. A control question seeks to identify a related behavior, but is not intended to elicit information regarding a factual issue. For example, the question “Are you presently sitting in a four-legged chair?” is easily and objectively verifiable, and thus serves to elicit an identifiable response. One or more control questions can be presented to a subject in conjunction with one or more relevant questions. Also as described above, a relevant question is intended to elicit information regarding a factual issue, but is not easily verifiable when presented separately. In combination, one or more control questions and one or more relevant questions provide a meaningful way to compare and interpret the relative strength of the physiological reactions of the subject.

Additionally, because a relevant question may be threatening to a subject, the subject may respond truthfully, but in a manner indicative of guilty knowledge or deceptive intent. As such, the one or more control questions can be used to develop an estimate of a deceptive response provided by the subject. A control question intended to elicit a deceptive response can be presented in a probable lie scenario, in which the subject presented with the question will most likely respond untruthfully. For example, the control question “During the first seventeen years of your life, did you ever do something dishonest or illegal?” can be presented to a subject. Such a control question, which will likely be answered deceptively, is deliberately vague and can be superficially related to one or more relevant issues. Further, a subject can be directed to answer the question deceptively or untruthfully. For example, a subject can be given a more directed control question to which they are instructed to respond with a lie, such as “During the first seventeen years of your life, did you ever violate even one rule or regulation?” An examiner can use the response to the one or more control questions to identify a pattern of reactions that correspond to deception and/or lying. The response to the one or more control questions then can be compared to a response to a relevant question to determine whether the reactions are sufficiently similar or different.

Regardless of the type of control question employed, it is anticipated that a physiological reaction by the same subject corresponding to a deceptive response to a relevant question will be more pronounced, as the subject will likely be concerned that the deception will be discovered. The reaction of a subject responding truthfully, however, is expected to be less pronounced than a deceptive response to a control question because the subject, when responding truthfully, may be concerned that the reaction corresponding to the deceptive response to the control question is not clearly differentiated from the reaction corresponding to one or more relevant questions.

If the subject demonstrates guilty knowledge or malicious intent, the subject can be asked to explain the positive result (110). If the subject fails to provide a satisfactory explanation, the subject can be identified for additional evaluation (112). If the subject provides a satisfactory explanation for a positive result, such as legitimate prior experience, the test can be terminated (116). The test also can be terminated (116) if a P-300 response was not detected in the subject after a sufficient number of relevant questions have been presented (114).

In another implementation, the ERP GKT can be designed to identify knowledge or recognition of specific information. For example, if the ERP GKT is intended to identify an individual with knowledge of terrorist activities or ties to a terrorist organization, the ERP GKT can include stimulus related to terror activities. In such an implementation, the ERP GKT can include images of known terrorist web sites, audio segments associated with known terrorist leaders, specific messages, images of specialized knowledge in handling and operating small arms and explosives, excerpts of Al-Qaeda's training in Taqiyya and Kitman, and text from selected Islamist authors.

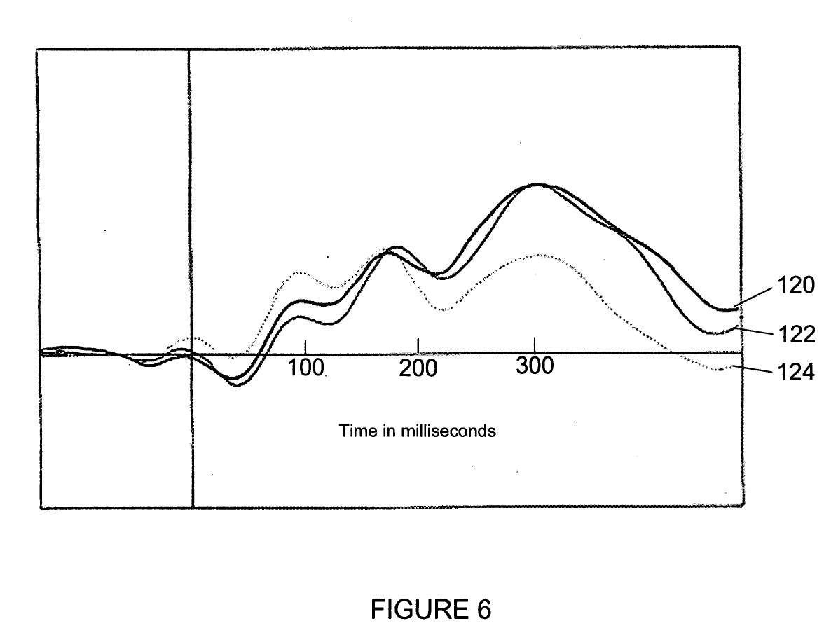

FIG. 6 depicts a wave form 120 detected by an EEG sensor associated with P-300 wave. The P-300 wave signifies the presentation (P) of a wave form corresponding to recognition approximately 300 milliseconds after the introduction of the stimulus. The detected P-300 wave form 120 is compared with a target wave form 122 that is indicative of guilty knowledge. Additional wave forms that are detected, such as the irrelevant wave form 124 represented by the dotted line, are discarded. As discussed above, the detection of the P-300 wave form indicates guilty knowledge and can be used to determine a need for additional lines of questioning as well as the time at which such additional lines of questioning should be pursued.

In another implementation, a plurality of multiple choice questions can be presented to the test subject, such as an Identity Verification Flow Chart (IVFC). The test subject can be instructed to answer each multiple choice questions as quickly as possible. Responses can be entered using a data entry device, such as one or more of the data entry devices 62 described above. The subject's responses to the IVFC can be measured by the headband 30 included in the helmet 10, which comprises the fNIR component. The question presentation and timing application can be configured to synchronize each verbal question with a set of multiple-choice answers that are presented to the subject. The multiple-choice answers can be presented using any display device known in the art, including those described above. For example, the display device can be the visual display 19 associated with the helmet 10, the one or more display devices 20 associated with the data processing device 12, or the display 54 included in the kiosk 52. Additionally, the one or more questions comprising the IVFC can be presented to the subject through the one or more microphones 17 included in the helmet 17. Alternatively, one or more of the questions comprising the IVFC can be presented through the one or more speakers 60 included in the kiosk 52, the visual display 19 associated with the helmet 10, the one or more display devices 20 associated with the data processing device 12, the display 54 included in the kiosk 52, or by another person, such as an examiner.

The test subject can be instructed to verbally respond to each question. A response to a question can be recorded using a microphone, such as the microphone 22 included in the helmet 10. If a question was first posed audibly, the same question can be subsequently displayed on the display device, and the subject can be instructed to select an appropriate response from a plurality of possible responses. For example, the subject can be presented with the question “Do you intend to answer each question truthfully?” The subject then can be instructed to respond either “Yes, I intend to answer each question truthfully” or “No, I do not intend to answer each question truthfully.” The IVFC can include a plurality of questions concerning the personal history and identity of the subject, including place of birth, nationality, birth date, given name, aliases, parents, and social security number. In another implementation, the ERP GKT can be presented in conjunction with the IVFC. Similarly, EEG data associated with one or more responses can be compared with corresponding fNIR data to further refine the detection of deception.

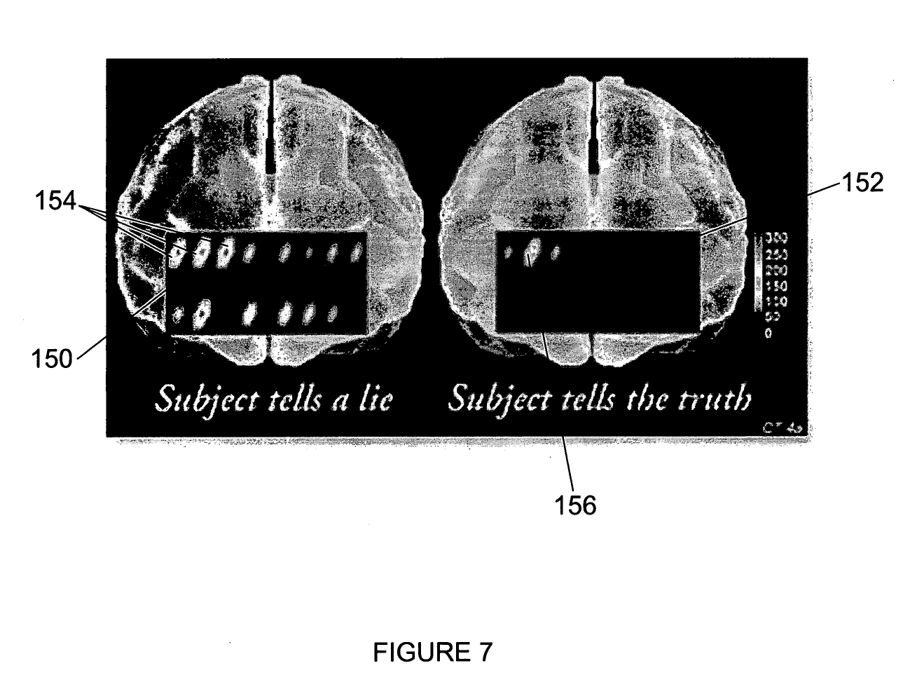

FIG. 7 presents the infrared light reflected back from the brain as sensed by one or more detectors, such as the silicon light detectors 36, during both a deceptive response 150 and a truthful response 152. When the deceptive response 150 is given, the one or more detectors indicate that a large amount of infrared light 154 is reflected back from the brain. Conversely, when the truthful response 152 is given, only a small amount of reflected infrared light 156 is detected by the one or more detectors. As such, the amount of reflected infrared light can be used to accurately determine whether the subject is providing a deceptive response.

In another implementation, either or both of the fNIR and EEG based tests can be combined with micro-expression analysis performed using an automated Facial Action Coding System (FACS). Because FACS is the least invasive form of testing, it also can be used before, after, or in conjunction with either or both of the ERP GKT and IVFC. The results of the micro-expression analysis can be analyzed in conjunction with the results of the ERP GKT and IVFC.

A computer configured to perform FACS analysis, such as the data processing device 12, can be configured to identify the occurrence of a subject attempting to conceal an emotion, rather than identifying an indication that a subject is providing a deceptive response. During FACS analysis, the facial features and facial structures of a subject can be analyzed using electronic markers that can identify even subtle movements. As such, the measured movements, when analyzed together or separately, can be used to identify whether an expression formed by a subject and the emotion underlying the expression, are genuine.

In FACS, micro-expressions are detected and analyzed. Because a genuine facial expression involves a plurality of muscles, a subject attempting to form a facial expression that is not genuine may not be able to recreate the essential elements associated with the genuine facial expression. A system configured to perform FACS analysis permits an observer, such as an examiner, to identify any discrepancies between a genuine expression and a forced expression. FACS functions by dividing a face into upper and lower facial movement. Further, FACS subdivides facial movements into action units, which comprise visibly discriminable muscle movements that can be combined to produce facial expressions.

Techniques for automatically recognizing facial actions in one or more sequences of images include analysis of facial movement through estimation of optical flow, holistic spatial analysis such as principal component analysis, independent component analysis, local feature analysis, linear discriminant analysis, and methods based on the outputs of local filters, such as Gabor wavelet representations and local principal components. The FACS application program can detect the presence of, or probability of, guilty knowledge and malicious intent in a subject. For example, analysis and detection of guilty knowledge and malicious intent can be based on individual expressions, such as curled lips, furrowed brows, muscle contractions, and expressions that begin and end in a jerky manner.

In order to combine FACS with either or both of the fNIR and EEG based tests, a camera can be integrated into one or more of the helmet 10, the data processing device 12, and the kiosk 52 to capture facial expressions and facial movements associated with the subject during one or more portions of a test, such as an ERP GKT, to identify guilty knowledge or malicious intent. In such an implementation, a FACS application program executed by the data processing device 12 or the processor 66 of the kiosk 52 can detect and analyze the subject's micro-expressions and interpret the results in conjunction with the results of either or both of the fNIR and EEG based tests.

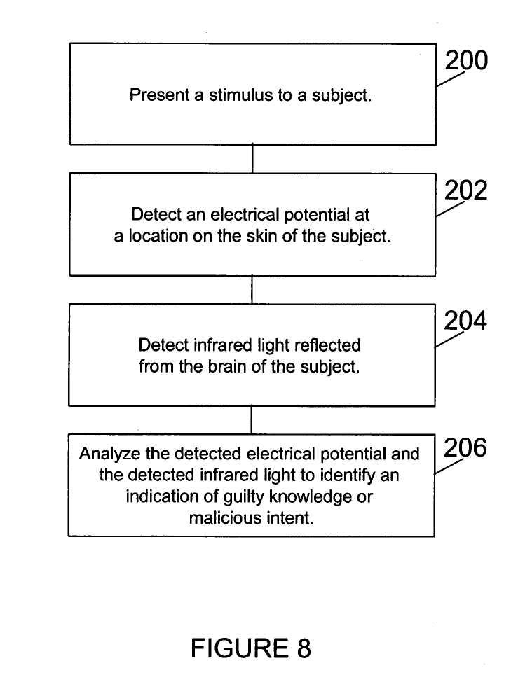

FIG. 8 describes a method of identifying guilty knowledge or malicious intent in a subject. In a first step 200, a stimulus is presented to a subject. In a second step 202, an electrical potential is detected at a location on the skin of the subject. In a third step 204, infrared light reflected from the brain of the subject is detected. Once the electrical potential and the reflected infrared light have been detected, the fourth step 206 is to analyze the detected electrical potential and the detected infrared light to identify an indication of guilty knowledge or malicious intent.

A number of implementations have been disclosed herein. Nevertheless, it will be understood that various modifications may be made without departing from the spirit and scope of the claims. Accordingly, other implementations are within the scope of the following claims.

What is claimed is:

1. A method of identifying guilty knowledge or malicious intent in a subject, the method comprising:
presenting a stimulus to a subject;
detecting an electrical potential at a location on the skin of the subject;
detecting infrared light reflected from the brain of the subject; and
analyzing the detected electrical potential and the detected infrared light to identify an indication of guilty knowledge or malicious intent.

2. The method of claim 1, further comprising:
associating the identified indication of guilty knowledge or malicious intent with the presented stimulus.

3. The method of claim 1, wherein the presented stimulus comprises a control question.

4. The method of claim 1, further comprising:
detecting one or more muscle movements comprising a facial expression; and
analyzing the one or more detected muscle movements to identify a visible indication of guilty knowledge or malicious intent.

5. The method of claim 4, further comprising:
associating the identified visible indication of guilty knowledge or malicious intent with the presented stimulus.

6. The method of claim 1, wherein the stimulus comprises one or more of a question, an image, a video clip, a sound, and an audio clip.

7. The method of claim 1, wherein detecting an electrical potential further comprises:
detecting a sensed electroencephalograph potential corresponding to a predetermined frequency band.

8. The method of claim 7, further comprising:
analyzing an amplitude associated with the sensed electroencephalograph potential; and
identifying a P-300 wave form.

9. The method of claim 1, wherein analyzing reflected infrared light further comprises:
determining an amount of reflected infrared light corresponding to one or more wavelengths; and
identifying a change in blood oxygenation based on the determined amount.

10. The method of claim 9, further comprising identifying a change in total blood volume based on the determined amount.

11. The method of claim 1, further comprising:
presenting an additional stimulus to the subject based on the identified indication of guilty knowledge or malicious intent;
detecting an electrical potential associated with the additional stimulus;
detecting reflected infrared light associated with the additional stimulus; and
analyzing the detected electrical potential and the detected infrared light to identify an indication of guilty knowledge or malicious intent associated with the additional stimulus.

12. A system for identifying guilty knowledge or malicious intent in a subject, the system comprising:
a stimulus presentation device;
a human wearable device including an EEG sensor and a light detector, wherein the human wearable device is configured to detect an electrical potential at a location on the skin of a subject and to detect infrared light reflected from the brain of the subject; and
a data processing device, wherein the data processing device is configured to analyze the detected electrical potential and the detected infrared light to identify an indication of guilty knowledge or malicious intent.

13. The system of claim 12, wherein the stimulus presentation device comprises one or more of a speaker and a visible display.

14. The system of claim 13, wherein the presentation device is physically coupled to the human wearable device.

15. The system of claim 12, wherein the human wearable device comprises a helmet.

16. The system of claim 12, wherein the human wearable device further comprises:
a light source comprised of a plurality of light emitting diodes, wherein two or more of the light emitting diodes produce light of different wavelengths; and
a modulator configured to modulate the wavelengths of light produced by the plurality of light emitting diodes.

17. The system of claim 12, further comprising:
a camera configured to capture a facial movement of the subject.

18. The system of claim 17, wherein the data processing device is further configured to:
analyze the captured facial movement to identify a visible indication of guilty knowledge or malicious intent.

19. The system of claim 12, further comprising a data entry device configured to receive an input from the subject.

20. The system of claim 19, wherein the data entry device comprises one or more of a keyboard, a mouse, a touch pad, a touch screen, a key pad, a microphone, a joystick, a button, a switch, a card reader, a scanner, and a biometric identification device.
